(19) United States
(12) Patent Application Publication
(10) Pub. No.: US 2014/0025020 A1
(43) Pub. Date: Jan. 23, 2014

(54) RADIATION SHIELDING CATHETER HUB AND RELATED METHODS OF USE

(71) Applicant: BOSTON SCIENTIFIC SCIMED, INC., MAPLE GROVE, MN (US)

(72) Inventor: NATHAN ZAMARRIPA, SUDBURY, MA (US)

(21) Appl. No.: 13/946,657

(22) Filed: Jul. 19, 2013

Related U.S. Application Data
(60) Provisional application No. 61/673,669, filed on Jul. 19, 2012.

(51) Int. Cl. A61B 6/10

(52) U.S. Cl. CPC A61B 6/107
USPC 604/263

(57) ABSTRACT

Catheter hubs adapted for use with medical devices as well as methods for making and using such catheter hub are disclosed. An example catheter hub may include a hub body having a proximal end, a distal end, and a lumen extending between the proximal end and the distal end. The proximal end of the hub body may include one or more ports and the distal end may be capable of attaching to a catheter shaft. The hub body may include a radiation shielding material.

### CROSS-REFERENCE TO RELATED APPLICATIONS

This application claims priority under 35 U.S.C. §119 to U.S. Provisional Application Ser. No. 61/673,669, filed Jul. 19, 2012, the entirety of which is incorporated herein by reference.

### TECHNICAL FIELD

This disclosure pertains generally to medical devices and medical procedures.

More particularly, the disclosure pertains to medical devices that include radiation shielding materials.

### BACKGROUND

Treatment regimens involving radiation therapy have become commonplace. For example, yttrium-90 (Y-90) intra-arterial radioembolization therapy is commonly employed in treating liver cancer. Generally, this treatment is delivered by loading radioactive Y-90 isotope on small beads, ranging in size from about 10 μm to about 50 μm. The beads, which may be referred to as microspheres, are delivered to the treatment site by a catheter inserted into a blood vessel and then navigated to the target location. Percutaneous delivery devices, however, do not provide for radiation shielding.

As a result, the physician performing the delivery procedure may be at risk for exposure to radiation. For example, an unshielded 5 mL syringe loaded with Y-90 therapeutics could expose a user to their annual radiation limit in less than one minute. Considering the equipment employed in a percutaneous delivery system, the catheter hub poses a particular problem. Not only does the catheter hub facilitate the attachment of delivery devices such as syringes, but that component is generally gripped by the physician during treatment and is handled by the physician and other personnel during disposal of the equipment.

Thus, there remains a need for improved radiation protection in the field of minimally invasive surgical devices used for delivering radiation therapeutics.

### SUMMARY

Medical devices as well as methods for making and using medical devices are disclosed. An example medical device may include a catheter hub. The catheter hub may include a hub body having a proximal end, a distal end, and a lumen extending between the proximal end and the distal end. The proximal end of the hub body may include one or more ports and the distal end may be capable of attaching to a catheter shaft. The hub body may include a radiation shielding material.

Another example medical device may include a catheter assembly. The catheter assembly may include a catheter shaft having a proximal end. A catheter hub may be attached to the proximal end of the catheter shaft. The catheter hub may include a proximal end, a distal end, and a lumen extending between the proximal end and the distal end. The catheter hub may include a radiation shielding material.

An example method for providing radiation shielding to a user during radiotherapy medical procedure may include advancing a catheter assembly through a body lumen to a position adjacent a target tissue. The catheter assembly may include a catheter shaft having a proximal end, a distal end, and a lumen defined therethrough. The method further includes attaching a catheter hub to the proximal end of the catheter shaft. The catheter hub may include a proximal end, a distal end, and a lumen extending therethrough. The hub further includes one or more ports at the proximal end for allowing access to the lumen of catheter shaft. The hub may include a radiation shielding material. The method includes advancing a radioactive agent through the hub and into the catheter shaft. The radioactive agent may then be advanced to a position adjacent the target tissue.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 is a schematic view illustrating an example catheter hub according to the present disclosure.

FIG. 2 is a schematic view illustrating a radiation shielded catheter assembly and a method of using the assembly according to the present disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. The following detailed description should be read with reference to the drawings in which similar elements in different drawings are numbered the same. The drawings, which are not necessarily to scale, depict illustrative embodiments and are not intended to limit the scope of the invention.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with one embodiment, it should be understood that such feature, structure, or characteristic may also be used connection with other embodiments whether or not explicitly described unless cleared stated to the contrary.

While the systems and methods described herein are discussed relative to cancer therapy using a radioactive agent, it is contemplated that the systems and methods may be used in other applications where radioactive agents are desired.

The present disclosure provides systems and methods that employ a radiation-shielded catheter assembly to inject a radioactive agent into a patient's body. To this end, the system may include a catheter assembly including a catheter hub and/or a catheter shaft made from a radiation shielding material. Manufacture of such catheter assembly may include an injection molding process. Other manufacturing processes such as extrusion, coating, or other suitable processes known to those skilled in the art may also be contemplated.

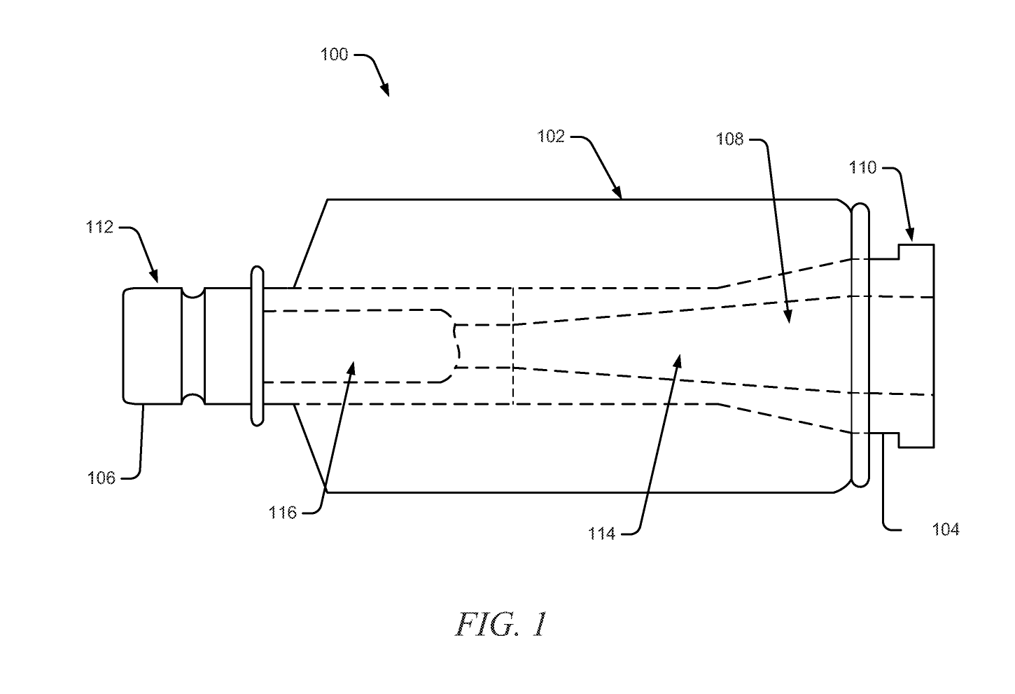

FIG. 1 is a schematic view of an example catheter hub 100 according to embodiments of the present disclosure. As shown, the catheter hub 100 may include a hub body 102 having a proximal end 104, a distal end 106, and a lumen 108 extending between the proximal end 104 and the distal end 106. The illustrated embodiment includes a generally cylindrical hub body, the distal end 106 being adapted for attachment to the remainder of the catheter and a proximal end 104 being adapted to facilitate attachment of auxiliary devices, as explained below. A person skilled in the art will appreciate that the hub 100 may include any suitable configuration, for instance, but not limited to, elliptical, cylindrical, circular, polygonal, irregular, or so forth. As shown, the hub 100 may include a wing-shaped configuration, for example including a pair of wings, which may provide enhanced gripping and comfort handling to the user.

In addition, catheter hub 100 may include a connector 112 disposed at the distal end 106 of the hub 100, which may connect to any suitable shaft member or a tube such as a catheter shaft (not shown). The proximal end 104 of the hub 100, however, may include a port 110 such as a hemostasis valve, a Toughy-Borst connector, or the like.

In some embodiments, lumen 108 may include one or more channels 114 providing passage to incoming therapeutic substances or devices. The channel 114 may be formed in a substantially funnel-like configuration with a tapered distal end, which may guide medical devices (e.g., a guidewire) through the catheter shaft. The channel 114 may be configured with cylindrical, irregular, or other suitable cross-sections known to those skilled in the art. In certain instances, the channel 114 may be in fluid communication with a lumen of the catheter shaft. In other embodiments, the hub 100 may employ a channel 116, extending between the distal end 106 and channel 114, providing a fluid communication path between those elements

Port 110 may couple to the proximal end 104 of the catheter hub 100 and may provide for attachment for one or more infusion devices, medical accessories, or the like in a well-known manner. As shown, the port 110 may be fixed to the proximal end 104 of the hub 100, which may also be disposed in a detachable manner when required. In the illustrated embodiments, the port 110 may include threads, which may provide for a detachable connection with an infusion device, such as—a syringe. While in other embodiments, the port 110 may include extending flanges, bayonets or other connection structures (not shown) useful with securing medical devices to the catheter hub 100. In addition, port 110 may define a channel or opening therethrough in fluid communication with the channel 114 and/or channel 116. The port 110 may define a substantially circular cross section or the like. Other suitable cross-sections, however, such as oval, cylindrical, irregular, or the like may also be utilized.

Connector 112, disposed at the distal end 106, may provide connection to the catheter shaft or other suitable accessories (not shown). In at least some embodiments, the catheter shaft (not shown) may be secured to the connector 112 permanently or temporarily. Connection mechanisms may include, but are not limited to, thermal bonding, adhesive bonding, mechanical bonding, snap-fit, threading, or other suitable structures. In one embodiment, the hub 100 may directly connect to the proximal end of the catheter shaft (not shown) using the connector 112. In certain instances, however, the hub 100 may employ a strain relief (not shown) connected integrally to the connector 112 at proximal end, such as the distal end of the strain relief may be disposed over a portion of the proximal region of the catheter shaft to substantially reduce strain that may occur at the connection point between the catheter shaft and the hub 100.

Conventionally, the delivery of radioactive agents to a target site for medical diagnoses and/or treatments may expose physician to harmful radiation. In addition, a catheter shaft (not shown) attached to the distal end of the catheter hub may expose a non-targeted tissue to emitted radiation through the catheter shaft, while navigating the catheter shaft within a patient body. The embodiments of the present disclosure provide a radiation shielding material be useful for reducing radiation exposure to the clinician and the patient. This may include the use of a radiation shielding material in the catheter hub 100, along portions or substantially the entire the length of the catheter shaft, or both.

The catheter hub 100 may include a variety of different materials. In at least some embodiments, the materials may include a composite made from one or more polymers and a radiation shielding material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, and the like.

The radiation shielding material may include a material that blocks or slows the passage of alpha, beta, and/or gamma radiation therethrough so that a user of the hub 100 may be shielded from such radiation. Some examples of radiation shielding material may include tungsten, bismuth trioxide, barium sulfate, combinations thereof, and the like, or other suitable materials.

In some embodiments, the radiation shielding material may be dispersed in or disposed throughout portions of the hub 100. Alternatively, one or more exterior surfaces of the hub 100 may include a coating that includes the radiation shielding material.

In an exemplary embodiment, the manufacturing method employed for the catheter hub 100 may include injection molding. However, this is not intended to be limiting. Other manufacturing methods may also be utilized such as, for example, extrusion or other suitable manufacturing methods.

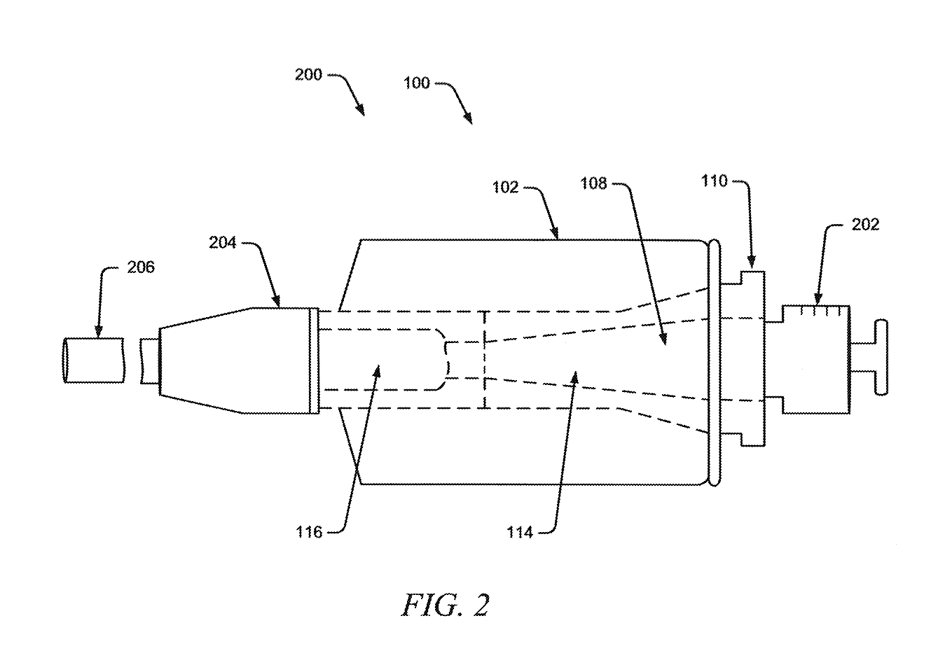

FIG. 2 is a schematic view of an exemplary radiation shielded catheter assembly 200 according to embodiments of the present disclosure. Radiation shielded catheter assembly 200 includes the catheter hub 100 (as shown in FIG. 1) coupled to a catheter shaft 206 at the distal end 106. Also shown is a strain relief 204 that is attached to the hub 100 and generally disposed about the distal end 106 of the hub 100. The strain relief 204 extends over a proximal portion of the catheter shaft 206 and may help to reduce strain that may be present at the connection point between the catheter shaft 206 and the hub 100.

The assembly 200 may be used to deliver a radioactive agent to or adjacent to a target tissue. The target tissue may vary. For example, in at least some embodiments the target tissue may be a diseased or cancerous tissue within the patient body. This may include a cancerous tissue of a body organ (e.g., liver, brain, stomach, small intestine, colon, bladder, kidney, lung, etc.) However, those skilled in the art will appreciate that the assembly 200 may be used to perform various other medical procedures known to those in the art, including imaging techniques or the like that may include radioactive agents.

In illustrated embodiments, catheter assembly may include a syringe 202 attached to the port 110. The syringe 202 may be used to pass the radioactive agent (not shown) into the hub 100 and into and/or through the catheter shaft 206 to a position at or adjacent to the target tissue. The precise form of the radioactive agent can vary. In at least some embodiments, the radioactive agent may include radioactive beads, gels, fluids, radioactive drugs, radioactive nucleotides, and/or nucleic acids, combinations thereof, and the like. For example, the radioactive agent may include beads including a radioisotope of yttrium such as yttrium-90. Other suitable radioactive agents are also be contemplated, without departing from the scope of the present disclosure including, for example, radioactive isotopes of technetium, rubidium, strontium, thallium, lutetium, iodine, boron, phosphorus, actinium, and the like.

The strain relief 204 may be formed from a suitable material (including those disclosed herein). The strain relief 204 may provide kink-resistance and support to the catheter shaft 206 near the hub 100. Furthermore, the strain relief 204 may define a passage to receive the catheter shaft 206 such that lumen through the hub 100 may be in fluid communication with the lumen of catheter shaft 206. In one embodiment, the strain relief 204 may be mechanically attached to the hub 100 or through any other suitable mechanism. As shown, the strain relief 204 may be tapered from its proximal end to the distal end. The strain relief 204 may also include other suitable cross-sections such as cylindrical, rectangular, oval, irregular, or other suitable shapes.

The method for using assembly 200 may begin with a clinician may introduce the distal end of the catheter shaft 206 within the patient body. The shaft 206 may be advanced through a body lumen (e.g., natural orifice, blood vessel, an incision, combinations thereof, etc.) to a position adjacent to a target tissue (e.g., cancer tissue). As disclosed herein, various parts of the catheter assembly 200 may include a radiation shielding material. For example, portions or the entire hub 100 may include a radiation shielding material. In some of these and in other embodiments, portions or all of catheter shaft 206 may include a radiation shielding material. In some of these and in other embodiments, portions or all of strain relief 204 may include a radiation shielding material. In some of these and in other embodiments, portions or all of infusion device 202 may include a radiation shielding material. In some of these and in other embodiments, portions or all of the distal tip of catheter 206 may include a radiation shielding material.

Although the embodiments described above have been set out in connection with a renal nerve ablation catheter, those of skill in the art will understand that the principles set out there can be applied to any catheter or endoscopic device where it is deemed advantageous to deflect the tip of the device. Conversely, constructional details, including manufacturing techniques and materials, are well within the understanding of those of skill in the art and have not been set out in any detail here. These and other modifications and variations my well within the scope of the present disclosure and can be envisioned and implemented by those of skill in the art.

Other embodiments of the present disclosure will be apparent to those skilled in the art from consideration of the specification and practice of the embodiments disclosed herein. It is intended that the specification and examples be considered as exemplary only, and departure in form and detail may be made without departing from the scope and spirit of the present disclosure as described in the following claims.

What is claimed is:

1. A catheter hub adapted for use with a medical device, the hub comprising:
a hub body having a proximal end, a distal end, and a lumen extending therebetween, wherein the proximal end of the hub body includes one or more ports and the distal end is capable of being attached to a catheter shaft;
wherein the hub body includes a radiation shielding material.

2. The catheter hub of claim 1, wherein the radiation shielding material is capable of shielding a user of the hub from yttrium-90.

3. The catheter hub of claim 1, wherein the radiation shielding material includes barium sulfate.

4. The catheter hub of claim 1, wherein the radiation shielding material includes bismuth trioxide.

5. The catheter hub of claim 1, wherein the radiation shielding material includes tungsten.

6. The catheter hub of claim 1, wherein the hub body includes a polymer-metal composite.

7. The catheter hub of claim 1, wherein the hub body is injection molded.

8. The catheter hub of claim 1, wherein the hub body is extruded.

9. A catheter assembly, comprising:
a catheter shaft having a proximal end; and
a catheter hub attached to the proximal end of the catheter shaft, the hub having a proximal end, a distal end, and a lumen extending therethrough;
wherein the hub includes a radiation shielding material.

10. The assembly of claim 9, wherein the catheter shaft includes a second radiation shielding material that is the same as or different from the radiation shielding material included with the hub.

11. The assembly of claim 9, wherein the hub includes a polymer-metal composite.

12. The assembly of claim 9, wherein the hub includes polyether block amide.

13. The assembly of claim 9, wherein the radiation shielding material is capable of shielding a user of the assembly from yttrium-90.

14. The assembly of claim 9, wherein the radiation shielding material includes barium sulfate.

15. The assembly of claim 9, wherein the radiation shielding material includes bismuth trioxide.

16. The assembly of claim 9, wherein the radiation shielding material includes tungsten.

17. A method for providing radiation shielding to a user during radiotherapy medical procedure, the method comprising:
advancing a catheter assembly through a body lumen to a position adjacent a target tissue, the assembly including:
a catheter shaft having a proximal end and a distal end, and a lumen defined therethrough; and
a catheter hub attached to the proximal end of the catheter shaft, the hub having a proximal end, a distal end, and a lumen extending therethrough, the hub further including one or more ports at the proximal end for allowing access to the lumen of catheter shaft wherein the hub includes a radiation shielding material,
advancing a radioactive agent through the hub and into the catheter shaft, and
advancing the radioactive agent through the catheter shaft to a location adjacent the target tissue.

18. The method of claim 17, wherein the catheter shaft includes a second radiation shielding material that is the same as or different from the radiation shielding material included with the hub.

19. The method of claim 17, wherein the radioactive agent includes yttrium-90.

20. The method of claim 17, wherein the target tissue is the liver.
